(19) United States
(12) Patent Application Publication
(10) Pub. No.: US 2011/0301513 A1
(43) Pub. Date: Dec. 8, 2011

(54) Dynamically Adjusted CPR Compression Parameters

(75) Inventor: Gary A. Freeman, Newton Center, MA (US)

(73) Assignee: ZOLL Medical Corporation

(21) Appl. No.: 13/152,054

(22) Filed: Jun. 2, 2011

Related U.S. Application Data
(60) Provisional application No. 61/350,865, filed on Jun. 2, 2010.

(51) Int. Cl. A61B 5/0452; A61M 5/00; A61H 31/00; A61B 5/026; A61N 1/39; A61N 1/18

(52) U.S. Cl. 601/43; 600/509; 607/5; 607/2; 600/301; 604/500

(57) ABSTRACT

A resuscitation system for use in resuscitation of cardiac arrest victims includes an ECG monitor programmed to monitor an organized, non-shockable rhythm of an electrocardiogram (ECG) signal from a patient undergoing lifesaving cardiac care; a processor programmed to identify a time during an electrocardiographic cycle of the ECG signal during which a vulnerable period for risk of fibrillation induction of the ECG will occur; and control circuitry for generating signals to cause a parameter descriptive of chest compressions to be modified so as to minimize risk of induction of fibrillation during the vulnerable period.

### CROSS-REFERENCE TO RELATED APPLICATION

This application claims priority to U.S. Provisional Application Ser. No. 61/350,865, filed on Jun. 2, 2010, entitled “Dynamically Adjusted CPR Compression Parameters,” the entire contents of which are hereby incorporated by reference.

### TECHNICAL FIELD

This document relates to systems and techniques for performing chest compressions on a victim of a cardiac abnormality, such as ventricular fibrillation, including by the use of mechanical chest compression apparatuses.

### BACKGROUND

Sudden health problems such as sudden cardiac arrest and injuries caused by accidents kill thousands of people and cause permanent injury every year. Fast and competent care to resuscitate such victims of these problems can be essential to positive outcomes in such situations. For example, it is said that the chance of surviving a sudden cardiac arrest falls by ten percent for every minute of delay in providing effective treatment.

Resuscitation treatments for patients suffering from cardiac arrest generally include clearing and opening the patient's airway, providing rescue breathing for the patient, and applying chest compressions to provide blood flow to the victim's heart, brain, and other vital organs. If the patient has a shockable heart rhythm (ventricular fibrillation or pulseless ventricular tachycardia), resuscitation also may include defibrillation therapy. Along with such action, an electrocardiogram (ECG) signal for the patient may be electronically captured, displayed, and monitored, so that rescuers can determine when the patient's heart has returned to normal or near-normal operation, and determine when the heart exhibits a shockable rhythm. About half of patients who suffer ventricular fibrillation (VF) have a recurrence of VF within minutes of successful VF conversion, which may then require reconversion. Patient odds of survival fall with repeated VF recurrence during resuscitation.

### SUMMARY

This document describes systems and techniques that may be used to provide improved chest compression for a patient or victim who is suffering from ventricular fibrillation or a similar malady. Specifically, a portion of a person's cardiac cycle that is in the area of the T wave as indicated by an ECG waveform, represents a vulnerable period of the heart's cycle. This is the portion of the heart's cycle during which the ventricles are repolarizing, and as discussed below, is a period in which external physical manipulation of the heart via chest compressions or decompressions can cause the heart to enter or re-enter ventricular fibrillation, especially when the heart has just been shocked out of fibrillation and into an organized rhythm like pulseless electrical activity (PEA). This period may be referenced as the vulnerable period.

By the techniques described here, motion of the heart via chest compressions is reduced or eliminated during the vulnerable period, such as by controlling the operation of a mechanical chest compression device in coordination with ECG readings received from a patient, so that movement for compressions or for decompressions is avoided during the vulnerable period. Also, the heart is particularly vulnerable just after it has received a large defibrillating shock or a drug such as epinephrine, and the techniques described here may avoid sudden compressions or decompressions particularly during such periods. For example, the velocity with which compressions or decompressions occur may be minimized just after a shock has been delivered to a patient, and may be increased as time elapses after the delivery of a shock and after the heart has recovered more from the shock.

In one implementation, a resuscitation system for use in resuscitation of cardiac arrest victims is disclosed. The system comprises an ECG monitor programmed to monitor an organized, non-shockable rhythm of an electrocardiogram (ECG) signal from a patient undergoing lifesaving cardiac care; a processor programmed to identify a time during an electrocardiographic cycle of the ECG signal during which a vulnerable period for risk of fibrillation induction of the ECG will occur; and control circuitry for generating signals to cause a parameter descriptive of chest compressions to be modified so as to minimize risk of induction of fibrillation during the vulnerable period.

In another implementation, a medical chest compression method is disclosed. The method comprises monitoring an electrocardiogram (ECG) signal of an electrocardiographic cycle from a patient undergoing lifesaving cardiac care, where the ECG signal represents an organized, non-shockable rhythm; determining a time during the electrocardiographic cycle in which a vulnerable period for risk of fibrillation induction of the ECG will occur; and generating signals to cause a parameter descriptive of chest compressions to be modified so as to minimize risk of induction of fibrillation during the vulnerable period.

Certain of the implementations discussed below may, in appropriate circumstances, provide one or more advantages. For example, a mechanical chest compression system may be controlled in a manner that delivers a sufficient volume of blood flow while lessening the chance that a patient's heart will refibrillate. Such control may be computer-mediated and automatic so as to improve its responsiveness and performance, and to allow human rescuers to focus their attention on other matters. Alternatively, such an approach may be applied to manual chest compressions, and under either automatic or manual compressions, it may improve a patient's chance of surviving an adverse cardiac event.

The details of one or more embodiments are set forth in the accompanying drawings and the description below. Other features and advantages will be apparent from the description and drawings, and from the claims.

### DESCRIPTION OF THE FIGURES

FIG. 1A shows a comparison of a single ECG cycle and possible chest compression profiles during the cycle.

FIG. 1B shows a comparison of an ECG waveform between defibrillating shocks and a variable chest compression profile.

FIG. 2 shows an example system, in schematic form, for providing dynamically controlled chest compression to a patient.

FIG. 3 is a flow chart of an example process for controlling chest compressions in coordination with an ECG waveform.

FIG. 4 is an activity diagram showing example operations for components of a lifesaving system.

FIG. 5 shows an example compression waveform having different velocities for downstroke and upstroke phases.

FIG. 6 shows a compression waveform in which depth during the at-depth phase is not held constant, but is gradually decreased so as to further minimize the upstroke velocity.

FIG. 7 is an example screenshot of a CPR feedback mechanism.

FIG. 8 is a schematic diagram of a general computing system that can be employed to operate a medical device in manners like those discussed here.

### DETAILED DESCRIPTION

This document describes mechanisms by which chest compressions for a patient suffering from sudden cardiac arrest can be coordinated with the patient's heart rhythm so as to lessen the chance that the patient will reenter cardiac fibrillation or another adverse cardiac state. In general, two distinct techniques may be applied either alone or in combination. First, the timing of physical compression and decompression movements may be coordinated with an ECG waveform so that such motions do not occur during the vulnerable period of the cardiac cycle or are minimized during such period. For example, a compression device may be controlled so as to not start a compression until after the vulnerable period has passed, or can hold a compression that has already begun until the end of a vulnerable period, and then release the compression, or can be synchronized to the R-wave of the ECG but reduce the duration of the compression so that the compression upstroke does not overlap the vulnerable T-wave. Second, the relative velocity with which compressions (whether in the downstroke or upstroke phase) occur can vary across an inter-shock time period. For example, the velocities may be a minimum just after the delivery of a shock, and much greater closer to the start of a next shock. In this manner, blood circulation may be maximized by providing greater pumping power at the end of a shocking cycle (and also thereby increasing the effectiveness of the next shock), while minimizing the interference with the heart at the beginning of the cycle when the heart is more vulnerable.

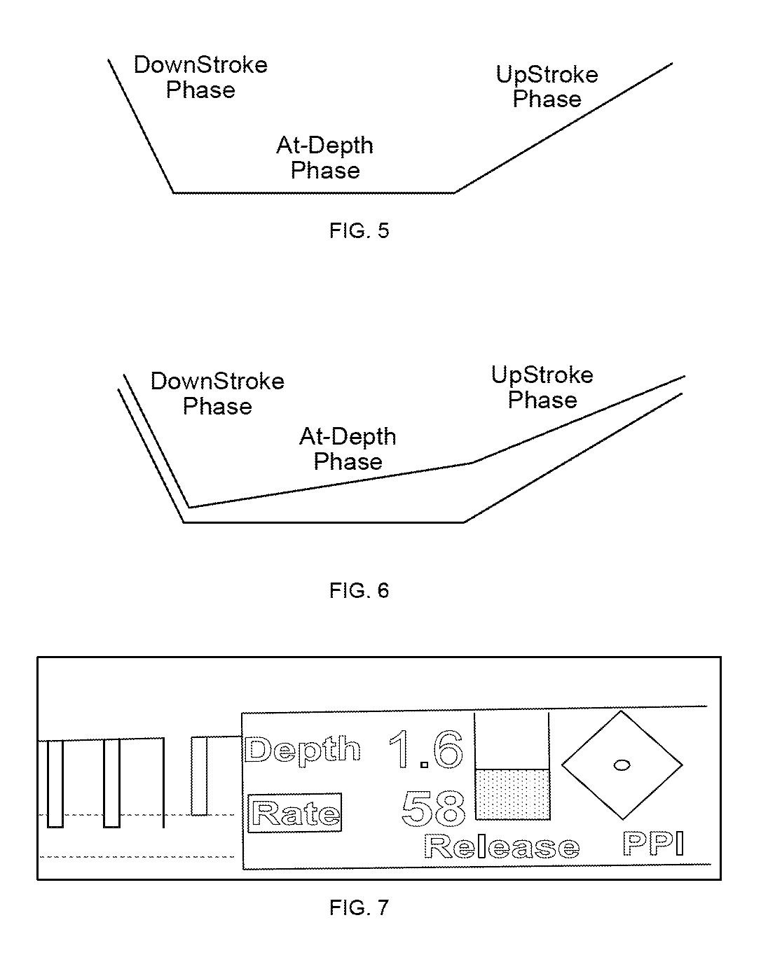

The velocities of the downstroke and upstroke can also be individually and independently adjusted so as to maximize blood flow while minimizing risk of refibrillation. For instance, by synchronizing the downstroke to the R-wave of the ECG, which is a period that is generally not vulnerable to refibrillation, the velocity of the downstroke can be maximized (e.g., with a downstroke duration of 25-150 milliseconds) to increase the duration of the portion of the compression cycle at which the depth is at a maximum, while independently decreasing the upstroke velocity (e.g., >100 milliseconds, and preferably >150 milliseconds) so that even though the upstroke may occur during the vulnerable T-wave, the risk of refibrillation is reduced. (see FIG. 5).

The depth of the at-depth phase may also be adjusted during the duration of the at-depth phase so as to minimize the depth at the time of the upstroke. This will result in lower velocity for the same upstroke duration, minimizing the risk of refibrillation (FIG. 6).

Such points may be implemented using a series of interrelated and inter-communicating medical devices. For example, a defibrillator may be connected to a patient via electrodes placed on the patient's chest so as to deliver a shocking electrical input to the patient in a familiar manner. Also, a mechanical chest compressor, such as in the form of a band that wraps around the patient's torso, may be applied, and may be responsive to signals received from the defibrillator and also from an ECG machine. Chest compression may also be performed manually by a rescuer, under prompting (e.g., spoken voice prompts and visual feedback on a display of a defibrillator) from a defibrillator or other device. In addition, other parameters of a patient may be tracked and integrated in determining the timing of, and velocity of, chest compressions for a patient. Each such medical device may also be combined with one or more of the other devices discussed here into a common housing for convenience and portability. Further details regarding these systems and techniques are described below.

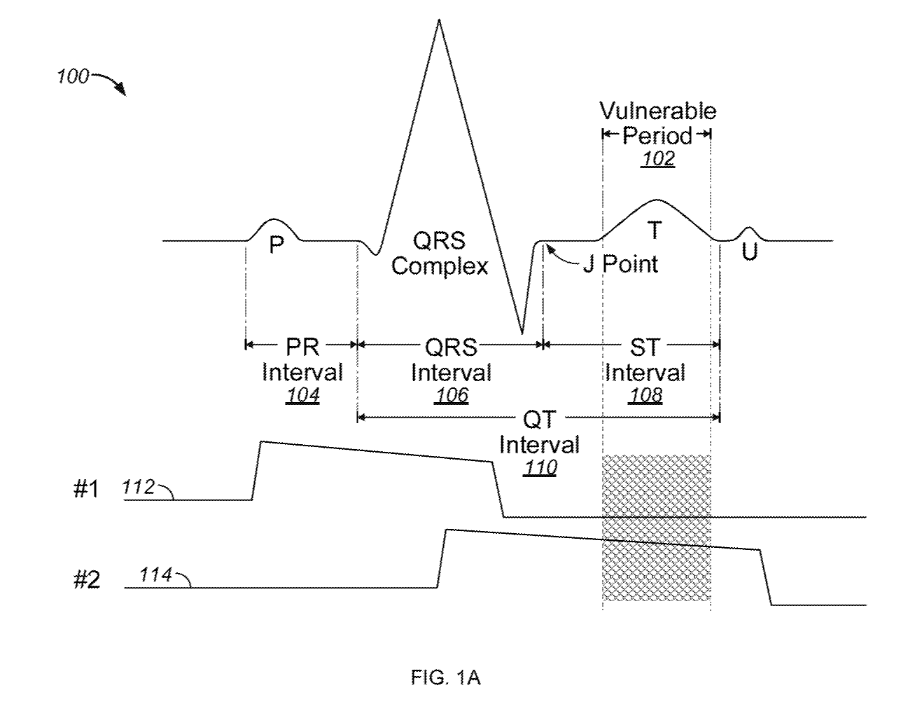

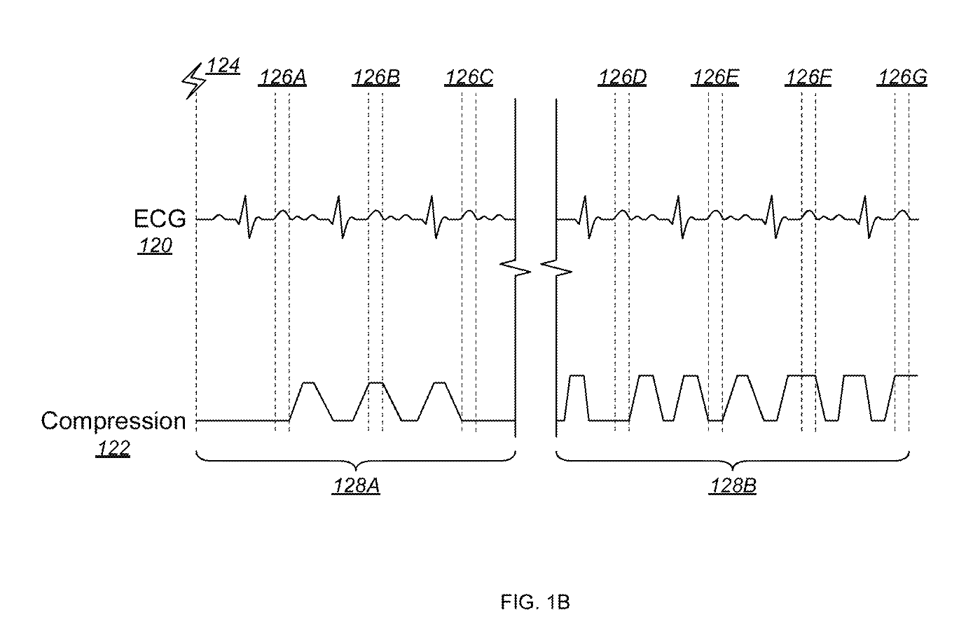

FIG. 1A shows a comparison 100 of a single ECG cycle and possible chest compression profiles that may be imposed during the cycle on a patient who generated the ECG signal. In general, what is displayed here is a timeline showing properties of the human heart that may be accommodated for, along with corresponding chest compression profiles that may be implemented so as to accommodate those properties. A typical cardiac ECG waveform for a single heartbeat is shown at the top of the figure. The waveform includes a PR interval 104, a QRS interval 106, and an ST interval 108—in familiar manner. Also, the QRS interval 106 (which includes a QRS complex) and the ST interval combined make up a QT interval 110. The ECG waveform or trace represents electrical activity of the heart that is captured by electrodes that are placed on the skin of a patient, such as via a typical 12-lead arrangement.

The PR interval 104 extends from the beginning of the P wave to the beginning of the QRS complex, and reflects the time an electrical pulse takes to travel from the heart's sinus node through its AV node and enter the ventricles. The QRS complex, and by extension the QRS interval 106, reflects the rapid depolarization of the heart's ventricles. The large amplitude of the waveform in this time zone reflects the relatively large muscle mass of the ventricles. The ST interval 108 is the period in which the ventricles are depolarized. The QT interval 110 is a combination of the QRS interval 106 and the ST interval 108. A prolonged QT interval is a risk factor for ventricular tachyarhythmias and sudden death.

The T wave, indicated by the letter T in the figure, and typically corresponding to vulnerable period 102, represents the repolarization or recovery of the ventricles.

The electrical changes represented by this ECG waveform are a function of complex chemical and mechanical changes that occur in the heart during each cardiac cycle or heartbeat. In particular, action of the heart muscle cells, or cardiomyocytes, to cause a heartbeat is the result of ordered propagation of excitatory stimuli that result in rapid depolarization and slow polarization of the heart muscle tissue. The cardiac action potentials that cause such action are the result of the flow of ions, or charged atoms, through ion channels, which are pores in the plasma membranes of cells that control the voltage gradient across the plasma membranes by allowing the flow of ions down their electrochemical gradient. For example, voltage-gated potassium channels in heart tissue open upon depolarization to allow efflux of potassium from the cells, and allow the repolarization of the membrane potential and to terminate an action potential. The proper flow of ions through other ion channels similarly affects the proper electrical status of those cells and the proper electrical activity in the heart, so that effective and coordinated pumping of the heart can occur.

Physical force applied to the heart tissue can affect the configuration of ion channels in the heart. For example, a sharp blow to the sternum, such as in the form of a precordial thump, is believed to cause certain ion channels to stay open or closed for a greater-than-normal time period, and to thus affect the depolarization or repolarization of the heart.

Likewise, commotio cordis is a process by which ventricular fibrillation can be induced by a sharp blow to the sternum of an individual who is not otherwise having cardiac problems. It may occur, though rarely, when an item like a baseball strikes a blow to a person's sternum. A person is generally subject to potential commotio cordis during a small part of the T wave portion of the cardiac cycle. It is possible that the physical impact associated with commotion cordis interferes with the natural opening and closing of the heart tissue's ion channels, thereby affecting the flow of ions through the channels, and in turn negatively affecting the ability of electrical excitation to pass in a proper coordinated manner through the heart tissue.

In one study (Link, Estes. Mechanically induced ventricular fibrillation (commotio cordis). Heart Rhythm, Vol 4, No 4, April 2007), it was found that the early phase of the T wave was the most vulnerable for a non-ischemic swine model. In the case of patients undergoing a cardiac arrest when the heart has undergone a significant period of ischemia as well as the insult of one or more high voltage defibrillation shocks, that period of vulnerability would be significantly expanded, in many cases, from what was observed in the Link study. Thus, the vulnerable window for fibrillation induction only roughly corresponds to the T-wave, and there will be varying probabilities of fibrillation induction within the sub-intervals during, immediately preceding, and subsequent to the T wave.

The two chest compression profiles 112, 114 represent techniques that result from an understanding that physical interference with the heart in or around the T wave can cause the heart to refibrillate or have similar adverse responses. The profiles 112, 114 each represent time on their X-axis that aligns with the time axis for the ECG, and represent a level of compression or decompression of a patient's chest, such as a number of inches of vertical motion of the sternum, or equivalent measure.

Profile 112 represents a compression that has been timed to avoid the vulnerable period 102 entirely. In particular, the compression starts at the beginning of the PR interval 104, holds while releasing somewhat through the PR interval and part of the QRS interval, and then releases. The profile 112 then stays released through the vulnerable period 102. The profile 112 is shown here for purposes of illustrating relevant timing of phases in the profile 112. While such a profile is illustrative, it would not be typical, as the rate of compressions is below 60 cycles per minute, and the compression is held longer than is typical. In actual operation, a second downstroke-upstroke cycle would typically be shown in the profile 112.

The same is true of profile 114. As illustrated in this example, however, the downstroke for profile 114 is slightly in advance of the vulnerable period 102. As such, there may not, as a practical matter, be time to complete an upstroke before the occurrence of the vulnerable period 102, in normal timing for chest compressions in a continuous and repeating cycle. As a result, the compression here has been held and extended through the vulnerable period 102, before the upstroke has begun. In each of the examples of profiles 112, 114, the determination of when to apply a downstroke and when to permit an upstroke may be made by monitoring the ECG signal, and coordinating the actuation of a chest compressor, or feedback for a rescuer, with the ECG signal so as to avoid or reduce compression motion (whether downstroke or upstroke or both) during the vulnerable period 102.

As it is generally not desirable to initiate compression during the P wave due to adverse hemodynamic effects, and it is most advantageous to initiate the downstroke during or shortly after the R wave, just as the heart is beginning its isovolumic contraction, the downstroke is preferably timed to occur shortly after the R wave. To achieve optimal hemodynamics, the at-depth duration should be on the order of 250 milliseconds, which may mean that the upstroke will occur at the same time as the vulnerable portion of the ECG. In such a case, the velocity of the upstroke can be independently decreased so that fibrillation will not result. As the downstroke phase of the compression occurs near the R wave, when the heart is relatively invulnerable to compression-induced fibrillation induction, the downstroke velocity may be kept significantly higher than that of the upstroke phase, if the upstroke phase occurs during the vulnerable period.

The compressions may, as noted, be synchronized to the ECG cycle of the patient when the compressions are being delivered manually by a caregiver. In such a situation, both the downstroke and upstroke velocities can be reduced below a threshold at which the risk of fibrillation induction during the vulnerable period is sufficiently reduced.

Thus, using such techniques, a rescuer may reduce the chance of a ventricular fibrillation induction occurring during the provision of cardiopulmonary resuscitation. The particular compression profile may be selected so as to provide adequate blood circulation while still avoiding or minimizing the motion of chest compressions during the vulnerable period.

FIG. 1B shows a comparison of an ECG waveform for the time period between defibrillating shocks and a variable chest compression profile for that period. In this example, multiple cardiac cycles are shown in time-wise alignment with a single example compression profile. The upstroke and downstroke phases are shown to avoid vulnerable periods entirely in this example, though such motion could occur during a vulnerable part of the cardiac cycle, though in a controlled manner such as with a velocity kept below a limit.

The cycles start at the left, where a defibrillating shock 124 has just been applied to the patient. An ECG 120 then shows a waveform for the patient's heart after that shock, and just before a subsequent shock that has not yet occurred, but would be off the right edge of the displayed traces. Break lines in the middle of the figure indicate that additional cardiac cycles will have occurred between the two shocks.

The compression profile example here shows both an effort to avoid downstroke or upstroke motion during vulnerable periods of the patient's cardiac cycle, and also efforts to change the velocity of downstrokes and upstrokes as a function of the heart's recovery from the shock over the course of the shock-to-shock period of the cycle. As an initial note, although a skilled artisan would understand that the ECG waveform 120 would come out of the shock in a less defined and repeated manner than what is shown in the figure, a standard normal ECG waveform is shown here for each heartbeat to better highlight the various parts of the waveform.

The compression profile has been constructed in various ways by a system in order to avoid adverse impacts on the patient's heart. For example, compression motion is reduced or avoided in all of the various vulnerable periods 126A-G. Reasons for doing so and mechanisms for doing so have been discussed above with respect to FIG. 1A. In particular, the ECG waveform 120 can be monitored and analyzed with known techniques to identify various portions of the waveform, and a prediction may be made to identify when in the future the particular portions of the waveform will be repeated.

A second point, which can be better understood by recognizing that the ECG waveform 120 and compression profile 122 are split into two portions down the middle of the figure (an initial portion 128A and a later portion 128B), is that the relative velocities of the compressions and decompressions have increased at the right in the figure compared to the left in the figure. Specifically, as shown in the profile 122, the slopes of the leading and trailing edges of the profile have steepened as time progresses after the shock 124, thus showing that the downstrokes and upstrokes are sharper (faster) further from the shock 124. Such higher downstroke and upstroke velocities that are reflected by the steeper edges may cause additional volumes of circulation compared to slower velocities, and thus may be generally preferable in all parts of the process, but may not be practicable just after the shock 124 has occurred, for risk of ventricular fibrillation (VF) induction. In addition, by the time of the right edge of the figure, it may be time for another shock, and the greater preparatory circulation of blood caused by the sharper motions may help that shock be more effective.

The velocity and depth may thus initially (after the particular shock) be set to a lower-than-optimal setting, with respect to best hemodynamics, and then gradually increased while monitoring the ECG for induced electrical activity that is the result of the downstroke or upstroke. This electrical activity is commonly referred to as an ectopic beat. It is typically these ectopic beats that occur during the vulnerable T wave that induce fibrillation. By gradually increasing the relevant parameters, the ectopic beats will be of reduced amplitude and thus be of lower risk for fibrillation. If an ectopic beat is detected as a result of either the downstroke or upstroke, that particular portion of the compression waveform can be adjusted without adversely affecting the other compression parameters.

Electrical stimulation may also be delivered to the heart at a lower level, such as in a pacing pulse, and the response of the heart to such an input may be monitored to determine the current condition of the heart, including its ability to avoid fibrillation around the vulnerable period. Such a pulse may be fired during the T wave phase, and intrinsic electrical activity of the heart that is induced by the pulse may then be monitored. The shape of such activity may inform a process regarding how the heart will react to inputs, and a compression profile, including compression velocities and/or timing may be affected using such information.

If an ectopic beat is triggered, the timing of the compression may be varied to determine what the time limits are of the vulnerable window for fibrillation induction.

Thus, by this understanding, a chest compression device can be controlled or a caregiver may be instructed, not only to avoid elevated compression velocities during the vulnerable periods, but also to provide less severe compression velocities while the heart is in its most fragile state, just after a shock has been provided to the heart by an external defibrillator. Such concerns may be accounted for while still providing adequate forceful compressions for the effective circulation of blood during CPR or other lifesaving procedures.

The profiles 112 and 114 in FIG. 1A and the profiles above in FIG. 1B show particular examples by which a compression profile may be altered from a traditional profile that is repeated in cycles, where the profile of each cycle matches the profile of previous and subsequent cycles. The relevant cycles can be compression/decompression cycles (FIG. 1A) or shock cycles that each envelop a plurality of compression/decompression cycles. While certain alternations of profiles have been discussed here, the profiles 112 and 114 may also be changed from one compression/decompression cycle to the next—e.g., by changing the timing of the start of a compression or decompression and/or by changing the speed of a compression or decompression or the length of a hold period (e.g., to drag it out past a vulnerable period)—via other alterations in a standard profile that may be set to be performed in the absence of identifying a need to avoid a vulnerable period. Similarly, other alterations than those shown above, may be provided during a period between shocks, though generally such alternations will still typically involve more aggressive action at times further after a shock than immediately after the shock.

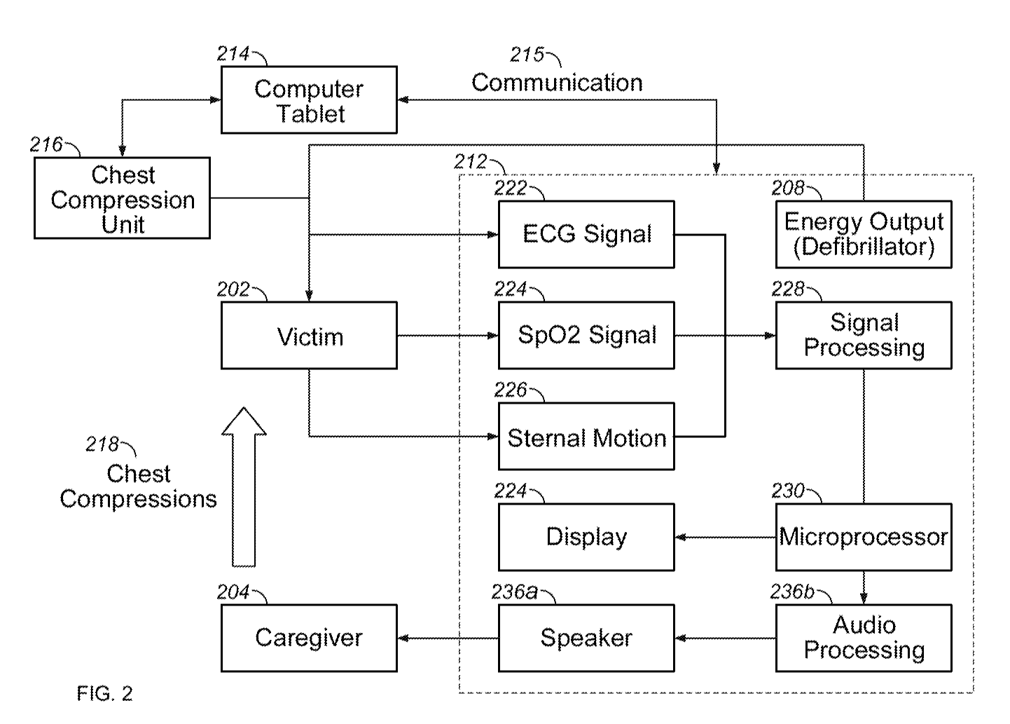

FIG. 2 shows an example system 200, in schematic form, for providing dynamically-controlled chest compression to a patient. In general, the system 200 involves a number of medical devices that may be used to provide life-saving care to a victim, such as a victim 202, of sudden cardiac arrest. The various devices may be part of a single unit or multiple units, and may be used to monitor various real-time physical parameters of the victim 202, to communicate between the components and with remote systems such as central caregivers, and to provide care to the victim 202 or provide instructions to caregivers, such as caregiver 204, in providing care to the victim 202.

The victim 202 in this example is an individual who has apparently undergone sudden cardiac arrest and is being treated by the caregiver 204. The caregiver 204 may be, for example, a civilian responder who has had limited training in lifesaving techniques, an emergency medical technician (EMT), a physician, or another medical professional. The caregiver 204 in this example may be acting alone or may be acting with assistance from one or more other caregivers, such as a partner EMT.

The victim 202 is in a position in which therapy has been provided to the victim 202. For example, a set of defibrillator electrodes 210 have been applied to the victim's torso in a typical manner and are in wired connection to a portable external defibrillator 208. The defibrillator 208 may be, for example, a typical automated external defibrillator (AED), a professional defibrillator, or other similar type of defibrillating apparatus. The victim 202 has also been provided with a ventilation bag 206, to provide forced air into the victim's lungs to assist in rescue breathing of the victim 202. The defibrillator 208 and ventilation bag 206 may be operated in familiar manners and in coordination by various caregivers. Also, the ventilation bag 206 may be fitted with various sensors and transmitters so as to communicate electronically with the defibrillator 208. For example, a volumetric flow sensor may be provided with the ventilation bag 206, and data about the volume of airflow to and from the victim may be passed to defibrillator 208, so the defibrillator 208 may relay such information, or may also use such information to affect the manner in which defibrillation is provided to the victim 202.

A computer tablet 214 is also shown communicating with the other devices, and being manipulated by caregiver 204. The tablet may serve as a general electronic command post for the caregiver 204 to receive information about the victim 202 and other items, to communicate with other caregivers, and to provide input in controlling the operation of the various components in the system 200. The tablet 214 may be provided with short range and long range wireless communication capabilities, such as Bluetooth or WiFi on the one hand, and cellular 3G or 4G on the other. The caregiver 204 may input information into the tablet computer 214, such as information describing the condition of the victim 202 and other similar information that is to be recognized and recorded by the caregiver 204. The tablet 214 may also be in data communication with multiple sensors for sensing real-time information about the victim 202, such as blood pressure, pulse, and similar real-time patient parameters. The caregiver 204 may also input information into tablet 214 so as to control one or more of the medical devices being used with the victim 202. For example, the user may adjust the type, intensity, speed, or coordination of treatment that is provided to the victim 202.

A Chest Compression Unit (CCU) 216 is one of the medical devices that may be provided for administering to the victim 202, either integrated physically with other devices or in a separate self-contained unit. In one implementation, the CCU 216 delivers the chest compressions via a load distributing band that is placed around a patient's upper thorax. The CCU 216 may take the form, for example, of the AUTOPULSE non-invasive cardiac support pump from ZOLL Medical Corporation of Chelmsford, Mass.. Such a device may be used to constrict the victim's chest evenly and thereby provide improved blood flow in the victim 202.

Various components within the Main Processing Unit (MPU) 212 may be employed to provide dynamically adjusted and potentially synchronized chest compressions with the CCU 216, where the compressions are coordinated in time with ECG waveforms and communicated to CCU 216.

A CCU 216 may not be available in certain situations, so that chest compressions 218 may also be delivered manually by the caregiver 204. In such a case, audiovisual feedback can be provided to the caregiver 204 via speaker 236a and display 224. Such feedback can direct the caregiver 204 to deliver compressions less forcefully when necessary, or at different speeds. It may also otherwise instruct the caregiver in the provision of care to the victim.

As shown in this example, multiple different input signals are received that characterize the current real-time condition or physical parameters of the victim 202. For example, an ECG signal 222 may be received by the MPU 212 and may represent current and real time ECG waveforms for the victim 202, which may be obtained by leads connected to defibrillator 208.

An SpO₂ signal 223, or other physiologically-derived signal that is either a direct or indirect measure of circulatory flow or perfusion, is also captured at box 224, and may be used to further determine when and at what force to apply chest compressions to the victim 202.

Although FIG. 2 shows specific examples of input signals such as SpO₂, an apparatus could use any relevant combination of physiological signals such as, but not limited to: ECG, measures of cardiac output, measures of heart rate, blood pressure(s), oxygen saturation (SpO₂), heart sounds (including phonocardiography), heart imaging (including ultrasound), and impedance cardiography. Compression parameters could use any relevant combination of features or measurements of compression including, but not limited to: compression velocity; compression depth; duty cycle; velocity of downstroke and upstroke; intrathoracic pressures during compressions; pleural pressures during compressions; sternal position, velocity or acceleration; chest wall or sternal strain or deformation; force applied to the chest; and pressure used to compress the chest by a mechanical chest compressor.

A sternal motion signal 226 is also sensed as an input to the MPU 212, such as to provide a feedback loop to determine the level of chest compression that has been provided to the victim 202. In particular, the MPU 212 may continue to supply a compression signal to the CCU 216 until feedback from the sternal motion signal 226 indicates that the compressions have achieved a particular degree of movement in the sternum (e.g., two inches of vertical motion).

The feedback based on the sternal motion signal 226 may also be used to feed back the amount of downstroke or upstroke velocity that a caregiver should be delivering so as to minimize the risk of inducing fibrillation. The feedback may be in the form of verbal prompts, e.g. “Release more slowly”, or a visual indicator via the display 224 (see FIG. 7) where the bar above the word “Release” will fill in proportion to the upstroke velocity of each compression delivered by the rescuer, but will turn red if the upstroke velocity is determined to be in excess of a limit above which the risk of induction of fibrillation is determined to be excessive. The goal of the rescuer is thus to maximize the filled portion of the “Release” bar without having it turn red. A coordinated combination of audible and visual feedback may also be provided.

A signal processing unit 228 is provided to filter inputs, such as ECG inputs, received from the patient for further analysis by the microprocessor 230. For example, the signal processing unit 228 may filter noise from input signals, and in the case of ECG data may filter artifacts created by chest compression motion of the victim 202 in order to remove such artifacts. Such preparation of ECG signals may be termed SEE-THRU CPR, and can be performed as discussed in U.S. Pat. No. 7,220,235, the teachings of which are incorporated herein by reference in their entirety.

A calculation may be performed using characteristics of the ECG, in particular the ST portion of the ECG waveform. For instance, ST elevation is indicative of ischemic injury and likely elevated sensitivity to compression-induced initiation of fibrillation. Based on the measurement of ST characteristics or other parameters of the ECG, the maximum safe thresholds for parameters of the compression—e.g. upstroke or downstroke velocity—can be calculated. The process for determining the maximum safe thresholds may also take into account state variables like numbers of shocks delivered, the amount of current or energy delivered for the immediately previous shock, a total amount of cumulative energy or current delivered for all shocks, particular morphological characteristics of the ECG such as T wave amplitude and inversion, QRS duration, and R wave curvature or sharpness.

Based on either retrospective data analysis of clinical datasets that include simultaneous recording of sternal motion signals and ECGs as well as patient outcome data, or of pre-clinical testing in animal models, a statistical model may have been developed that can predict the risk of fibrillation induction. The statistical model may in turn be used to determine an appropriate compression profile to be provided to a patient, and to be aligned in time with ECG data of the patient. The statistical model may be in the form of either a linear or non-linear regression equation, using such techniques as multiple logistic regression.

There may be multiple inputs to the regression equation, such as compression depth, upstroke and downstroke velocity, and the timing of each of the compression phases relative to the T-wave, as well as resuscitation information like defibrillation energy, number of shocks, etc., or ECG information such as ST elevation, T-wave amplitude etc. as mentioned above, thus forming an input vector. The regression equation will thus form a matrix calculation, where the input vector, X, is a 1×n dimensional matrix where n is the number of input variables, and the regression transformation matrix is an n×n matrix. The output vector, Y, is a 1×n matrix where each element is the probability that that particular parameter will induce fibrillation.

The input matrix may only incorporate ECG, other physiological signals like SpO2, or other perfusion measure and resuscitation information elements, to form a 1×p matrix with p elements, a transformation matrix of dimension p×q, where q are the number of compression parameters to be optimized, e.g. upstroke and downstroke velocity at-depth duration, etc. Based on the a priori-derived statistics and a theoretical model of the effect of compression parameters on blood flow, optimal control methods known to those skilled in the art may be employed, such as Hamiltonian control theory as introduced by Pontryagin or as an alternative, Gauss, Radau, or Lobatto pseudospectral optimal control, to achieve maximum blood flow while minimizing risk of fibrillation induction.

The theoretical model of the effect of compression parameters on blood flow may be a mathematical description of the circulatory system, such as that described in Crit Care Med 2000 Vol. 28, No. 11 (Suppl.). As that article describes, a system of differential equations has been described in a number of publications. In the specific instance of the article, “the human circulation is represented by seven compliant chambers, connected by resistances through which blood may flow. The compliances correspond to the thoracic aorta, abdominal aorta, superior vena cava and right heart, abdominal and lower extremity veins, carotid arteries, and jugular veins. In addition, the chest compartment contains a pump representing the pulmonary vascular and left heart compliances. This pump may be configured to function either as a heart-like cardiac pump, in which applied pressure squeezes blood from the heart itself through the aortic valve, or as a global thoracic pressure pump, in which applied pressure squeezes blood from the pulmonary vascular bed, through the left heart, and into the periphery. Values for physiologic variables describing a textbook normal “70-kg man” are used to specify compliances and resistances in the model. The distribution of vascular conductances (1/resistances) into cranial, thoracic, and caudal components reflects textbook distributions of cardiac output to various body regions.” The input to the model is then the real time sternal motion waveform during chest compressions.

A microprocessor/analyzer 230 is provided to receive input information regarding the real-time parameters of the patient, including ECG waveform data, and to perform analysis on such data to cause, and dynamically adjust, compressions and decompressions to be executed by CCU 216 or to modify the feedback prompts provided on the display 224 and annunciated by the speaker 236a. In this example, the coordination is between (a) an ECG waveform, (b) optional shocking by the defibrillator 208, and (c) provision of signals to the CCU 216 to cause the CCU 216 to actuate in coordination with the ECG in the manners discussed above and below. Thus, for example, the Main MPU 212 may receive the ECG signal 222, may monitor the ECG signal to determine the rate of repetition of the signal (i.e., the patient's cardiac cycle) and the current location in the signal, and may use such information to compute the time boundaries for the next vulnerable period. Using such boundaries, the microprocessor/analyzer 230, executing stored software code, may compute a compression profile to be applied to the victim 202 so that compression/decompression motion of the CCU 216 is avoided during the vulnerable periods for the victim 202, and/or so that the velocity of motion is decreased in the period soon after a shock has been provided, and increased at a time farther after the shock has been provided, and closer to the time before a next shock is to be provided.

Triggering circuits 238 may be signaled by the MPU 212 at appropriate times so as to generate the signals needed to cause the CCU 216 to squeeze and release the victim 202. Sternal motion signals 226 may be captured during such a process so that the MPU 212 can determine the level of compression or decompression that has been achieved, and can adjust the signals sent to the CCU 216 accordingly, using familiar closed loop control techniques.

The process carried out by the system 200, then, may be a continuous and cyclic process in which ECG data flows into the MPU 212 and is processed so that a continuous series of chest compressions may be provided to the victim 202 and coordinated with shocks from the defibrillator 208 until the caregiver 204 intervenes (e.g., by changing certain parameters or by ending the process, such as when the victim 202 has restored his or her normal or sustainable heart rhythm). Particular ones of the chest compressions may differ from particular other ones in their compression profiles, including in the relative timing of the start of a compression or decompression, the rate of the compression or decompression, the length of hold times between compressions and decompressions, and in the overall length of a compression cycle.

The chest compression actions and/or other actions taken with respect to the victim 202 may also be taken manually and may be prompted by the various devices, including the MPU 212. Each of the changes in compression profile discussed above may also be prompted via such a manual process. For example, coordinated chest compression times and rates may be computed by the microprocessor/analyzer and may be verbally announced by the audio processor/speaker unit 236a and 236b in familiar manners. For example, a metronome of beeping sounds may be played to indicate to the caregiver 204 during CPR when to press down on the victim's 202 chest so as to avoid compressions or decompressions during a vulnerable period. Also, a display 224 may provide coordinated visual feedback, such as by showing an ECG waveform, showing a graph of compression depth for the victim 202, and showing other similar data that may be helpful in aiding the caregiver 204 in providing for the victim 202.

Because sudden changes in the pace of a metronome may be disconcerting for someone performing manual chest compressions, the predictive process may “look forward” by multiple compression cycles in order to identify particular areas, such as vulnerable areas, in the waveform several beats ahead. The system may then gradually change the pace of the metronome at a rate that allows the rescuer to keep up with the changes, and still places the compressions/decompressions at particular locations vis-à-vis the heart waveform and at particular speeds.

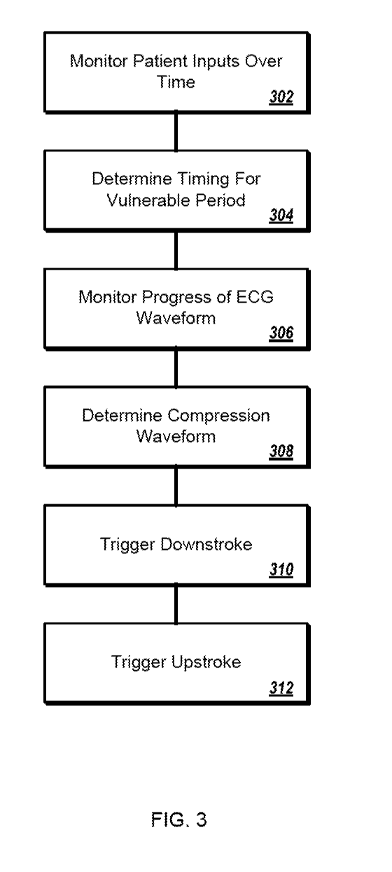

FIG. 3 is a flow chart of an example process for controlling chest compressions in coordination with an ECG waveform. In general, the process is a repeating and continuing process by which ECG data from a patient is monitored and analyzed so as to provide chest compressions to the patient in a manner that avoids compression or decompression velocities that may tend to cause refibrillation or other problems with the patient's heart.

The process begins at box 302, where one or more patient inputs are monitored over a period of time. Such inputs may include an ECG waveform or data representative of the ECG activity in the patient's heart, among other possible inputs from the patient. The ECG waveform may be of a typical form involving increases and decreases in voltage that are computed from, for example, a 12-lead system in a familiar manner.

At box 304, the process determines the timing for the T wave portion of the ECG waveform (for an upcoming cycle or upcoming portion of a cycle). Such a determination may be made in various familiar ways including by matching real-time incoming ECG data to a profile of a typical ECG waveform over a cardiac cycle and determining the start and end of the waveform in addition to particular points along the waveform. Such a determination may be made across multiple cardiac cycles, so as to determine an average waveform, and thus extend this average form (which may be maintained as a running average over n prior cycles) into the future. For example, after a heart has been shocked with the defibrillator, the features of the waveform may be less pronounced and may begin to trend back to their normal characteristics over time. The identification of features in the waveform may look to multiple such cycles, and may be updated over time as the waveform returns to its normal status, rate, and shape.

Such monitoring and analysis may be continuous, and trend lines or other techniques may be used to predict where such features of the waveform will appear for future cardiac cycles. For example, a determination may be made regarding the probable length of the next cardiac cycle, and the relative position of the vulnerable zone within that cycle. The boundaries of the coming vulnerable zone may thus be computed from such observations.

Thus, at box 306, the progress of the ECG waveform is monitored so as to enable the computation of a future time or times during which a vulnerable period will occur. Using such information, a compression waveform is determined at box 308. The compression waveform defines when, and at what velocity and displacement, a chest compression device will be actuated for the patient in coordination with the ECG waveform, or a caregiver will be instructed or provided feedback in performing CPR. The compression waveform or profile is computed so as to provide an appropriate level of circulation in the patient while avoiding compression velocities that are timed relative to the ECG waveform when the heart may be likely to induce re-fibrillation in the patient.

At box 310, downstrokes and upstrokes are triggered by the process following the computed compression waveform. Such triggering may be part of a continuous process, where the ECG waveform is monitored and future timing for the ECG waveform is determined, and a compression waveform is computed to align with the ECG waveform in the manners discussed here. Also, the compression may be triggered in similar manners, as shown at box 312, and may have its velocity slowed or its occurrence delayed or accelerated to avoid velocities that could induce ventricular fibrillation (VF).

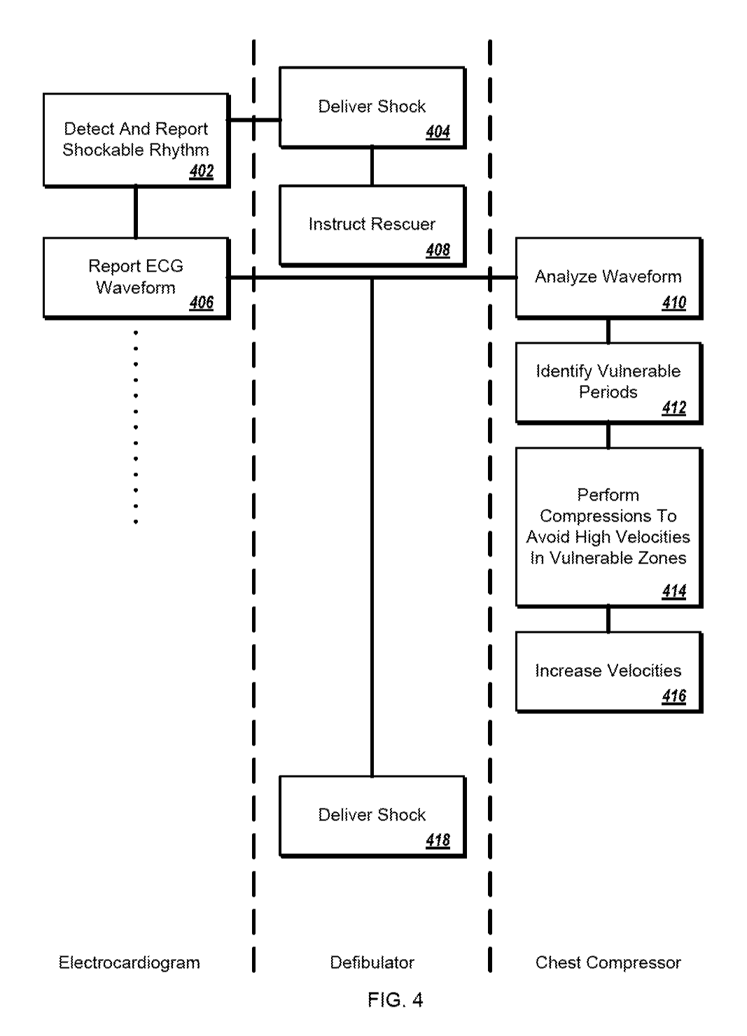

FIG. 4 is an activity diagram showing example operations for components of a lifesaving system. In general, this diagram shows particular actions that may be taken in an example configuration by different medical devices used at the scene of a medical emergency. In the example, the devices include an electrocardiogram device that generates a data representation of an electrocardiogram signal from a patient, a defibrillator which analyzes various parameters of a patient and generates electrical shocks to defibrillate the patient, and a chest compressor, which is a mechanical device used to apply automatic mechanical chest compressions to a victim in coordination with the other devices treating the patient. The process may be performed, for example, by a system such as system 200 in FIG. 2, and references are made here to the system for purposes of illustration.

The process begins at box 402, where the electrocardiogram function of the MPU 212 detects and reports a shockable rhythm in a victim's heart. The defibrillator 208 may then deliver the shock at box 404 in an effort to return the victim's cardiac function to a normal and sustainable state. Such shocking of the heart may have a substantial effect on the ECG waveform for the heart, which may be picked up by the electrocardiogram function of the MPU 212 and recorded in an ongoing representation of the ECG waveform at box 406. As indicated by a dotted line, such reporting of the waveform may be continuous throughout the process. Data from the MPU 212 may be obtained by any device that establishes communication with the electrocardiogram device, such as by forming a wireless short-range data communication link 215 to a device such as the tablet 214 (which may be a general tablet computing device that is loaded with an application for communicating with and managing the other components in the system).

At box 408, the MPU 212 begins instructing the rescuer in instructing CPR actions (which instructions also would have been occurring before the shock was provided). Such instructions may include spoken commands from the MPU 212 instructing the rescuer to perform rescue breathing, to stay away from the victim, and other similar instructions. At the same time, at box 410, the MPU 212 is analyzing the ECG waveform. Such analysis may take a variety of forms including efforts to lock onto the waveform and to determine times at which different portions of the waveform will occur in future cycles. Such analysis may thus lead to an identification of a vulnerable period for each cycle, such as by identifying the characteristics of the T wave and then identifying parts of the waveform in or around the T wave that can be used to determine a vulnerable period.

At box 414, the CCU 216 performs chest compressions, in a manner that is coordinated to avoid high chest movement velocities during the relevant vulnerable zones that have been computed by the process. Typically, the processing of the ECG will take place within the MPU 212 and, based on that processing, the MPU 212 will send control signals to the CCU 216 to adjust its compression parameters. The processing could also be distributed, and the MPU 212 could send the ECG and other relevant patient and physiological data to the CCU 216, which will then process the ECG data to determine optimal compression parameters.

In addition, as the patient travels further and further in time from the electrical shock provided in box 404, the velocities of downstrokes and upstrokes may be increased (box 416) so as to provide even greater hemodynamic effect to the patient from the CCU 216 or caregiver 204. Finally, at box 418, the MPU 212 determines that an additional shock is needed (and that the patient has a shockable rhythm), and delivers the shock while coordinating with the other components. For example, the CCU 216 may be halted automatically while the shock is provided and then may be restarted after the shock is delivered.

The process shown here can then repeat in a continuous manner, with the MPU 212 reacquiring signals for activity by the heart and analyzing those signals to determine the rhythm of the cardiac cycle again and to determine locations in time in which compressions should be conducted more gradually or not at all in order to minimize the chance of negative disruptions to the heart's rhythm.

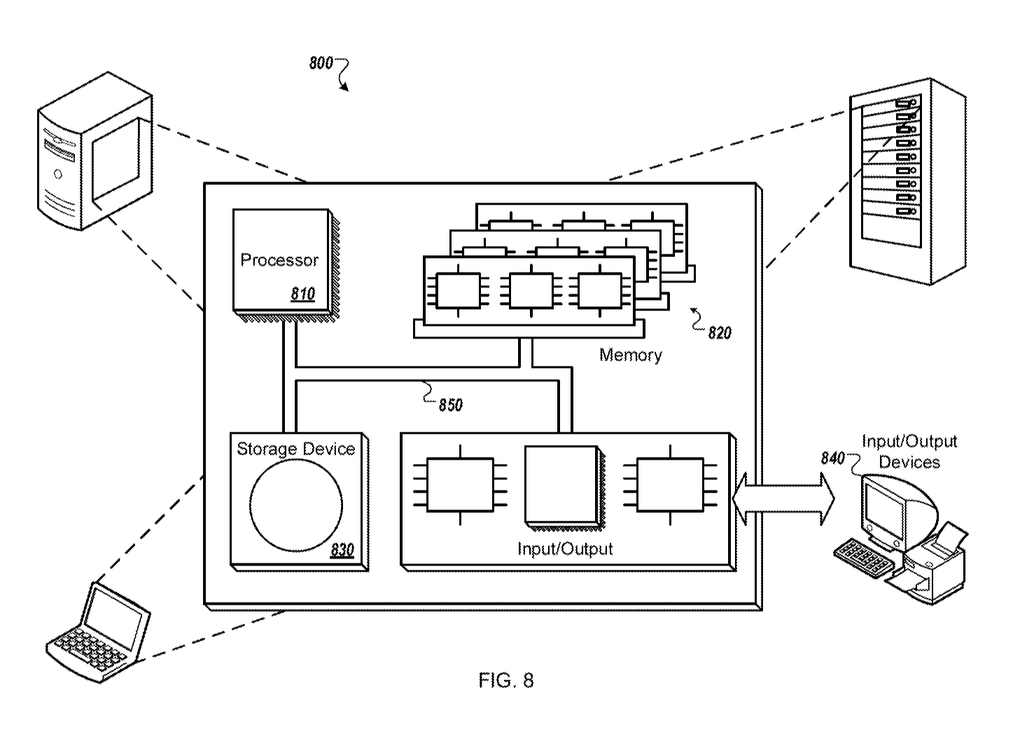

FIG. 8 is a schematic diagram of a general computing system 800 that can be employed to operate a medical device in manners like those discussed here. The system 800 can be used for the operations described in association with any of the computer-implement methods described previously, according to one implementation. The system 800 includes a processor 810, a memory 820, a storage device 830, and an input/output device 840. Each of the components 810, 820, 830, and 840 are interconnected using a system bus 850. The processor 810 is capable of processing instructions for execution within the system 800. In one implementation, the processor 810 is a single-threaded processor. In another implementation, the processor 810 is a multi-threaded processor. The processor 810 is capable of processing instructions stored in the memory 820 or on the storage device 830 to display graphical information for a user interface on the input/output device 840.

The memory 820 stores information within the system 800. In one implementation, the memory 820 is a computer-readable medium. In one implementation, the memory 820 is a volatile memory unit. In another implementation, the memory 820 is a non-volatile memory unit.

The storage device 830 is capable of providing mass storage for the system 800. In one implementation, the storage device 830 is a computer-readable medium. In various different implementations, the storage device 830 may be a floppy disk device, a hard disk device, an optical disk device, or a tape device.

The input/output device 840 provides input/output operations for the system 800. In one implementation, the input/output device 840 includes a keyboard and/or pointing device. In another implementation, the input/output device 840 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device, for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

The computer system may include software for implementing an electronic patient care record, for example the ePCR software of ZOLL Data Systems (Broomfield Colo.). The software provides the ability to enter, store and transmit patient information as well as therapeutic interactions. The computer is often a so-called “tablet” computer system that has been ruggedized for pre-hospital use, but may also take the form of an IPHONE or IPAD. Data is preferably transmitted in real time between the portable “tablet” computer to an MPU 212, such as data that indicates the delivery of epinephrine to a victim. As epinephrine may increase risk of VF induction, notification of its delivery may be used by the MPU to adjust the compression parameters to further minimize risk of VF induction. Other separate treatments provided to the patient, or parameters of the patient condition sensed by the various sensors may also be provided to the tablet, and may factor into the rate, timing, force, or speed with which compressions and decompressions are performed on the patient.

A number of embodiments have been described. Nevertheless, it will be understood that various modifications may be made without departing from the spirit and scope of the invention. Accordingly, other embodiments are within the scope of the following claims.

What is claimed is:

1. A resuscitation system for use in resuscitation of cardiac arrest victims, the system comprising:
an ECG monitor programmed to monitor for an organized, non-shockable rhythm of an electrocardiogram (ECG) signal from a patient undergoing lifesaving cardiac care;
a processor programmed to identify a time during an electrocardiographic cycle of the ECG signal during which a vulnerable period for risk of fibrillation induction of the ECG will occur; and
control circuitry for generating signals to cause a parameter descriptive of chest compressions that is to be performed on the patient to be determined so as to reduce a risk of induction of fibrillation during the vulnerable period.

2. A medical chest compression method, comprising:
monitoring an electrocardiogram (ECG) signal of an electrocardiographic cycle from a patient undergoing lifesaving cardiac care, wherein the ECG signal represents an organized, non-shockable rhythm;
determining a time during the electrocardiographic cycle in which a vulnerable period for risk of fibrillation induction of the ECG signal will occur; and
generating, with a computer-controlled medical device, signals to cause a parameter descriptive of chest compressions that are to be applied to the patient, to be set so as to reduce risk of induction of fibrillation during the vulnerable period.

3. The method of claim 2, where the parameter comprises downstroke velocity to be applied to the patient's chest.

4. The method of claim 2, wherein the parameter comprises upstroke velocity to be applied to the patient's chest.

5. The method of claim 2, wherein the parameter comprises a change in compression depth during an at-depth phase of compression of the patient's chest.

6. The method of claim 2, where the signals are generated following a defibrillation shock.

7. The method of claim 2, wherein the vulnerable period is during at least some portion of a T wave for the patient.

8. The method of claim 2, wherein the signals are generated as a result of identified morphological features of the ECG signal.

9. The method of claim 8, where the morphological features comprise ST segment deviations.

10. The method of claim 2, further comprising monitoring one or more physiological signals indicative of blood flow in addition to the ECG signal, and modifying parameters descriptive of chest compressions to be performed on the patient so as to minimize risk of fibrillation induction while maximizing blood flow for the patient.

11. The method of claim 10, wherein the modification of parameters is based on optimal control theory.

12. The method of claim 2, wherein the parameter descriptive of chest compressions comprises a downstroke velocity, and the generated signals are directed toward causing the downstroke velocity to be below 16 inches per second.

13. The method of claim 12, wherein the generated signals are directed toward causing the downstroke velocity to be below 13 inches per second.

14. The method of claim 13, wherein the generated signals are directed toward causing the downstroke velocity to be below 10 inches per second.

15. The method of claim 2, wherein the parameter descriptive of chest compressions comprises an upstroke velocity, and the generated signals are directed toward causing the upstroke velocity to be below 16 inches per second.

16. The method of claim 15, wherein the generated signals are directed toward causing the upstroke velocity to be below 13 inches per second.

17. The method of claim 15, wherein the generated signals are directed toward causing the upstroke velocity to be below 10 inches per second.

18. The method of claim 2, further comprising synchronizing a downstroke to occur within 150 milliseconds of occurrence of an R wave in an ECG cycle.

19. The method of claim 2, further comprising causing an upstroke to be delayed until after at least 25% of a T wave has occurred in an ECG cycle.

20. The method of claim 2, further comprising:
increasing the patient from a level of the parameter that has a low likelihood of inducing fibrillation;
monitoring the ECG signal for the occurrence of ectopic electrical activity induced by compression of the patient's chest; and
decreasing the parameter that is descriptive of chest compressions if ectopic activity is detected.

21. The method of claim 2, where the generated signal creates feedback to a rescuer performing chest compressions on the patient.

22. The method of claim 21, wherein the generated signal provides feedback to guide upstroke velocity.

23. The method of claim 21, wherein the feedback comprises audio feedback.

24. The method of claim 21, wherein the feedback comprises visual feedback.

25. The method of claim 2, wherein the generated signal comprises one or more control signals that control chest compression parameters of a computer-controlled mechanical chest compression device.

26. The method of claim 25, wherein the chest compression device includes a housing that is separate from a housing that contains an ECG processor that processes the ECG signal.

27. The method of claim 26, wherein the control signals are transmitted wirelessly to the chest compression device.

28. The method of claim 25, wherein the control signals are transmitted via a cable to the chest compression device.

29. The method of claim 28, wherein the chest compression device is integrated with a housing that contains ECG circuitry for monitoring the ECG signal.

30. The method of claim 2, further comprising using a data entry device, that is separate from a medical device that monitors the ECG signal, to notify the medical device in real time of initiation of a therapeutic intervention that is determined to potentially heighten a risk of fibrillation, and further comprising factoring a heightened risk into a determination of the parameter descriptive of chest compressions to be applied to the patient.

31. The method of claim 2, further comprising automatically, and in coordination with causing the parameter descriptive of chest compressions that are to be applied to the patient, directing application of an intervention with the patient that comprises a delivery of a vasopressor such as epinephrine or vasopressin.

32. The method of claim 2, wherein the computer-controlled medical device comprises an electromagnetic therapeutic energy generator arranged to deliver a therapeutic level of energy to the patient.

33. The method of claim 2, further comprising causing a mechanical chest compression device to be actuated to compress a patient's chest so that motion of the mechanical chest compression device avoids the vulnerable period of the ECG.

34. The method of claim 2, wherein monitoring the ECG signal comprises filtering chest compression motion artifacts from the ECG signal received from electrodes attached to the patient.

35. The method of claim 2, further comprising changing a profile for generating the signals during an inter-shock period between delivering defibrillation shocks to the patient.

36. The method of claim 35, wherein changing the profile comprises changing downstroke velocity or upstroke velocity across the inter-shock period.

37. The method of claim 36, wherein changing the downstroke or upstroke velocity comprises increasing velocity later in the inter-shock period in comparison to velocity earlier in the inter-shock period.

38. The method of claim 2, further comprising directing at-depth phase of chest compression of the patient through a vulnerable period to be held, and delaying upstroke until after the vulnerable period has passed.

39. The method of claim 2, wherein determining a time during the electrocardiographic cycle in which a vulnerable period for risk of fibrillation induction of the ECG signal will occur comprises identifying times for at least two ECG cycles to determine an ECG period, and determining a time during which the vulnerable period will occur by extending a period from a vulnerable period of one of the at least two ECG cycles.

40. A chest compression system, comprising:
an ECG analyzer programmed to identify one or more portions of an ECG waveform for a patient, including a period of the ECG waveform during which the patient's heart is particularly vulnerable to chest compression induced fibrillation, and to generate one or more signals that direct the occurrence of chest compressions so as to avoid chest motion during the vulnerable period; and
triggering circuitry arranged to receive a signal from the ECG analyzer and to cause instructions to be issued for upstroke, downstroke, or upstroke and downstroke of a patient chest in coordination with the received signal.

41. The system of claim 40, further comprising a chest compressor arranged to provide automatic chest compressions to a patient suffering cardiac arrest in response to direction from the triggering circuitry.

42. The system of claim 41, wherein the chest compressor comprises a belt arranged to be wrapped around the patient's chest and to squeeze in on the patient's chest when it is actuated.

43. The system of claim 40, further comprising a signal processing unit arranged to filter chest compression motion artifacts from an ECG signal received form electrodes attached to the patient.

44. The system of claim 40, wherein the analyzer is further programmed to change a profile for generating the signals during an inter-shock period between delivering defibrillation shocks to the patient.

45. The system of claim 44, wherein changing the profile comprises changing a velocity of downstroke motion or upstroke motion across the inter-shock period.

46. The system of claim 45, wherein changing the velocity of downstroke motion or upstroke motion comprises increasing the velocity later in the inter-shock period compared to a velocity earlier in the inter-shock period.

47. The system of claim 45, further comprising changing a rate of compressions during the inter-shock period.

48. The system of claim 40, wherein the analyzer is further programmed to hold the at-depth phase of the chest compression through a period around a T wave period of the patient and to delay an upstroke until after the vulnerable period has passed.

49. The system of claim 40, wherein the analyzer is programmed to determine a time during which a T wave portion of the ECG will occur, by identifying times for at least two ECG cycles to determine an ECG period, and determining the time during which the T wave portion will occur by extending the period from a T wave of one of the at least two ECG cycles.

50. A chest compression system, comprising:
a chest compression actuator arranged to provide automatic chest compressions to a patient suffering cardiac arrest using a chest compressor;
triggering circuitry connected to the chest compression actuator to provide signals to coordinate operation of the chest compression actuator; and
means for analyzing an ECG waveform of the patient to provide triggering signals to the triggering circuitry
an ECG analyzer programmed to identify one or more portions of an ECG waveform for the patient, including a period during which the patient's heart is particularly vulnerable to induction of fibrillation due to chest compressions, and to generate one or more signals that provide for motion of the chest compressor so as to avoid motion during the vulnerable period; and
triggering circuitry arranged to receive a signal from the ECG analyzer and to cause upstroke and downstroke phases of the chest compressor in coordination with the received signal.
